(19) United States
(12) Patent Application Publication
(10) Pub. No.: US 2024/0329053 A1
(43) Pub. Date: Oct. 3, 2024

(54) A CELL-BASED METHOD FOR DETECTING POTENTIALLY PATHOGENIC AUTOANTIBODIES TO NEURONAL NICOTINIC ACETYLCHOLINE RECEPTORS

(71) Applicant: TZARTOS NEURODIAGNOSTICS, Athens (GR)

(72) Inventors: John TZARTOS, Cholargos (GR); Socrates TZARTOS, Kaisariani (GR)

(21) Appl. No.: 18/293,087

(22) PCT Filed: Aug. 2, 2022
(86) PCT No.: PCT/IL2022/050838

§ 371 Date: Jan. 29, 2024

Related U.S. Application Data
(60) Provisional application No. 63/228,283, filed on Aug. 2, 2021.

(51) Int. Cl. G01N 33/68; G01N 1/30

(52) U.S. Cl. CPC G01N 33/6854; G01N 1/30; G01N 33/6893; G01N 2001/305

(57) ABSTRACT

Cell-based method for detecting antibodies against subunits of the neuronal nicotinic acetylcholine receptors in a subject is disclosed. The method comprises steps of
engineering cells to express at least one protein;
optionally, culturing said cells with at least one substance;
exposing said cells to body fluid or tissue from subject; and
detecting a reaction between at least one protein and antibodies found in body fluid or tissue,
obtaining a diagnosis for the patient.

A kit is also disclosed for detecting antibodies against subunits of the nicotinic acetylcholine receptors in a subject. The kit comprises cells expressing at least one protein; at least one medium for cell culture, at least one buffer for cell culture; and optionally, at least one substance.

### FIELD OF THE INVENTION

The present disclosure relates to the field of medicine and molecular biology. More particularly, the present disclosure relates to a diagnostic cell-based method for the detection of antibodies against subunits of the neuronal nicotinic acetylcholine receptors in patients with autoimmune autonomic ganglionopathy and other neuroimmune diseases related to nicotinic receptors.

### BACKGROUND OF THE INVENTION

Neuronal nicotinic acetylcholine receptors (nAChR) are a group of homopentameric or heteropentameric cationic channels, formed by nine α (α2-α10) and three β (β2-β4) subunits. nAChRs are ubiquitously expressed in the central and peripheral nervous system, modulating neurotransmitter release or mediating postsynaptic neurotransmission. nAchR dysfunction has been described in a broad spectrum of neurological diseases, including Parkinson's disease, Alzheimer's disease, autism and schizophrenia. In the peripheral nervous system, nAChRs are linked with dysautonomia of autoimmune cause, specifically autoimmune autonomic ganglionopathy (AAG), with antibodies against the α3-subunit containing nAChRs (α3-nAChRs).

In vivo and in vitro studies indicate that antibodies against α3-nAChRs may have pathogenic properties resulting in deterioration of synaptic transmission at the sympathetic, parasympathetic, and enteric ganglia (see “Lennon et al., 2003” and “Vernino et al., 2004”). Such antibodies have been found in the sera of patients with AAG characterized by autonomic failure, accompanied by the following main clinical features: orthostatic hypotension, xerostomia, pupillary dysfunction, urinary retention, anhidrosis, and gastrointestinal dysmotility. However, in several studies using the classical antibody detection techniques, such as radioimmunoprecipitation, low titers of antibodies against α3-nAChRs have also been detected in a broad spectrum of other diseases rather than AAG, such as postural tachycardia syndrome, diabetic autonomic neuropathy, autoimmune encephalitis, paraneoplastic disorders, particularly in association with small cell lung carcinoma, and more. Those findings indicate that the commonly used techniques for diagnosing AAG are not sufficiently specific, and may lead to false or unclear results.

The currently established and widely used method for the detection of antibodies against α3-nAChRs is a radioimmunoprecipitation assay (RIPA) with I125-epibatidine labeled α3-nAChR. Although this method is commonly used in diagnosis and treatment of AAG, it has a major drawback: low antibody titers have low specificity for AAG (˜50%), frequently identified in patients with various diseases, such as postural orthostatic tachycardia syndrome (POTS), diabetic autonomic neuropathy and patients with encephalopathy, where the role of these antibodies has not yet been fully determined.

In view of the prior art and given the various challenges described above, there is still an unmet long-felt need to produee a diagnostic method which is uniquely designed for the detection of only potentially pathogenic antibodies to neuronal nAChRs and disease-specific, with main emphasis on the AAG-specific antibodies. Cell based assay (CBA) is the gold standard method currently used for the detection of potential pathogenic antibodies against neuronal and glial antigens, usually with high disease-specificity. The present invention utilizes said method for the detection of disease-specific antibodies to neuronal nAChRs.

### BRIEF DESCRIPTION OF THE FIGURES

The accompanying drawings, which are included to provide a further understanding of the invention and are incorporated in and constitute a part of this specification, illustrate embodiments of the invention and together with the description serve to explain the principles of the invention.

FIG. 1 depicting fluorescence images showing the effect of chaperons and nicotine on α3β4 and α3β2 nAChR expression and the successful use of the CBA of the present invention.

FIG. 2 depicting fluorescence images showing the CBA of the present invention with α3β4 and α3β2 nAChR transfected HEK293 cells, detecting antibodies in sera with high or low RIPA-titers only in AAG patients and not in POTS patients.

FIG. 3 depicting a graphical presentation of CBA antibody titers for α3β4-nAChR compared to RIPA antibody titers for α384-nAChR; and

FIG. 4 depicting fluorescence images showing the CBA of the present invention with α4β2 nAChR transfected HEK293 cells (and AQP4-transfected cells as controls), detecting antibodies in a serum of a patient with autoimmune encephalitis

### SUMMARY OF THE INVENTION

It is an object of the present invention to disclose a cell-based method for detecting antibodies against subunits of the neuronal nicotinic acetylcholine receptors in a subject, comprising steps of:
a. engineering cells to express at least one protein;
b. optionally, culturing said cells with at least one substance;
c. exposing said cells to body fluid or tissue from said subject; and
d. detecting a reaction between said at least one protein and said antibodies found in said body fluid or tissue,
thereby, obtaining a diagnosis for said patient.

It is another object of the present invention to disclose the method of the above, wherein said antibodies against subunits of neuronal-type nicotinic acetylcholine receptors are selected from a group consisting of IgG, IgA, IgM and any combination thereof.

It is another object of the present invention to disclose the method of the above, wherein said subunits are subunits of neuronal nicotinic acetylcholine receptors.

It is another object of the present invention to disclose the method of the above, wherein said subunits are selected from a group consisting of α3, α4, α5, α7, β2, β4 and any combination thereof.

It is another object of the present invention to disclose the method of the above, wherein said subject suffers a neurological disorder.

It is another object of the present invention to disclose the method of the above, wherein said disorder is autoimmune autonomic ganglionopathy.

It is another object of the present invention to disclose the method of the above, wherein said engineering is condueted by means of molecular biology or genetic engineering.

It is another object of the present invention to disclose the method of the above, wherein said cells are selected from a group consisting of rodent cells, simian cells, human cells, diseased cells, transgenic cells and any combination thereof.

It is another object of the present invention to disclose the method of the above, wherein said at least one protein is selected from a group consisting of subunits of nicotinic acetylcholine receptors, chaperons, and any combination thereof.

It is another object of the present invention to disclose the method of the above, wherein said subunits of nicotinic acetylcholine receptors are subunits of ganglionic nicotinic acetylcholine receptors.

It is another object of the present invention to disclose the method of the above, wherein said at least one substance is a substance configured to activate, trigger, inhibit or modify the activity or the expression levels of said at least one protein.

It is another object of the present invention to disclose the method of the above, wherein said at least one substance is selected from a group consisting of nicotine, acetylcholine, epibatidine, cytisine, varenicline, anatoxin-α, azetidine, altinicline, lobeline, epiboxidine, passetidine, alobelin, arecoline, metacholine, xanomeline, carbamylocholine, cevimeline, pilocarpine and any combination thereof.

It is another object of the present invention to disclose the method of the above, wherein said body fluid or tissue selected from a group consisting of blood, serum, cerebrospinal fluid, lymph, urine, sweat, saliva and any combination thereof.

It is another object of the present invention to disclose the method of the above, wherein said detecting a reaction is condueted by means of molecular biology, immunofluorescence, radioactivity, luminescence and any combination thereof.

It is another object of the present invention to disclose a kit for detecting antibodies against subunits of the nicotinic acetylcholine receptors in a subject, comprising:
a. cells expressing at least one protein;
b. at least one medium for cell culture,
c. at least one buffer for cell culture; and
d. optionally, at least one substance.

It is another object of the present invention to disclose the kit of the above, wherein said cells are selected from a group consisting of rodent cells, simian cells, human cells, diseased cells, transgenic cells and any combination thereof.

It is another object of the present invention to disclose the method of the above, wherein said at least one protein is selected from a group consisting of subunits of nicotinic acetylcholine receptors, chaperons, and any combination thereof.

It is another object of the present invention to disclose the method of the above, wherein said at least one substance is a substance configured to activate, trigger, inhibit or modify the activity or the expression levels of said at least one protein.

It is another object of the present invention to disclose the method of the above, wherein said cells are fixed with methanol, paraformaldehyde or other fixatives.

It is another object of the present invention to disclose a cell harboring a vector for use in detecting of antibodies against subunits of the nicotinic acetylcholine receptors.

### DETAILED DESCRIPTION OF THE PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a sensitive cell-based method for the disease-specific antibodies to neuronal nAChRs.

As used herein after, the term “about” refers to any value being up to 25% lower or greater than the defined measure.

As used herein after, the term “autoimmune autonomic ganglionopathy/AAG” refers to a rare neuronal disease, which is a form of dysautonomia. The immune system of AAG patients produees antibodies targeting the neuronal (ganglionic) nicotinic acetylcholine receptor (nAChR), thus, inhibiting ganglionic AChR currents and impairing transmission in autonomic ganglia. The symptoms of this disease vary and may include: gastrointestinal dysmotility, constipation, diarrhea, anhidrosis, neurogenic bladder, severe orthostatic hypotension, pupillary dysfunction, syncope, chronic dryness of the eyes and mouth and more. The cause for AAG can be idiopathic, in which the body produees antibodies which attack ganglionic nAChR for unknown reasons, or paraneoplastic, in which antibodies are produeed against a tumor. AAG can be diagnosed using blood tests designed for the detection of the anti-ganglionic nAChR antibodies. However, the antibodies' levels are usually detected in only about 50% of AAG patients. The seronegative AAG patients (those without detectable AChR antibody levels) are speculated to have one or more different antibodies responsible for this neurological disease. Treatment of AAG usually involves immunosuppressive agents and corticosteroids.

As used herein after, the term “nicotinic acetylcholine receptors/nAChRs” refers to receptors made of several subunits of polypeptides, which respond and bind to the neurotransmitter acetylcholine, and therefore are referred to as cholinergic receptors. These receptors can also bind nicotine and its various analogues. The nAChRs are found in the autonomic nervous system, the neuromuscular junction, the brain, immune cells, lung cells and various other tissues. The nAChRs are non-selective cation channels, meaning that several different positively charged ions can cross through them. The channel is permeable to sodium and potassium, and some subunit combinations are also permeable to calcium. The movement of cations causes a depolarization of the plasma membrane, leading to an excitatory postsynaptic potential in neurons, and the activation of voltage-gated ion channels. AChRs in vertebrates are formed by one or more subunits selected from a pool of α1-10, β1-4, γ, δ, ε subunits Also, the entry of calcium to the neurons acts on different intracellular cascades and can trigger gene expression. Several different nAChRs have been identified that can be distinguished into neuronal-type (central nervous system and ganglionic) and muscle-type nAChRs.

As used herein after, the term “neuronal nAChRs” refers to nAChRs made of five subunits selected from α2-10 and β2-4 nAChR subunits. The autonomic ganglionic nAChRs prevalently comprise two as subunits and three β₄ subunits (but several other combinations including the α₅, α₇ and β₂ subunits are known in the literature), and they are located at the autonomic ganglia (a cluster of nerve cell bodies in the autonomic nerve system). In AAG patients, the immune system produees antibodies which specifically target the α3 subunit, therefore, they are referred to as anti-α3-nAChRs antibodies, anti-AChRα3 antibodies or anti-Nicotinic Acetylcholine Receptor α3 antibodies. Antibodies to CNS nAChRs (like α4β2 and α7 nAChRs) could be involved in autoimmune encephalitis similarly with antibodies to other CNS receptors like the NMDA receptor.

As used herein after, the term “radioimmunoprecipitation assay/RIPA” refers to a laboratory technique using the principles of immunoprecipitation (the precipitating of a protein antigen [coupled with radioactivity] from a solution using an antibody that specifically binds to that particular protein). In other words, this assay entails the coupling of a radioactive agent to the antigen in question, so once this radio-labeled antigen binds to specific antibodies, a detectable radioactive precipitate is formed, which can be further quantified into numerical or other values. In a non-binding manner, when the present application compares RIPA with the novel cell-based assay of the present invention, it may refer to the coupling of I125-epibatidine to α3-nAChR, but other radioactive agents may be used interchangeably.

As used herein after, the term “cell-based assay/CBA” refers to any assay that uses a cellular platform. This cellular platform might be based on plant cells, insect cells, avian cells, rodent cells, and mammalian cells, preferably simian or human cells or any other cell type. Moreover, the cells can originate from healthy, uncompromised tissues, or from diseased tissues, and they can be transfected or genetically modified or edited. These types of assays are widely used in the medical, biological and pharmacological fields, and they can be harnessed for example, for investigating biochemical activities and mechanisms, cytotoxicity, drug interactions, cell viability, high-throughput screening of compounds, immunological assays (such as antibody detection systems) and more. In the context of the present invention, the cell-based assay comprises cells expressing subunits of the nicotinic acetylcholine receptors with or without other relevant proteins (such as chaperons), and is meant to replace other technique commonly used for the detection of anti-α3-nAChRs antibodies in AAG patients, named radioimmunoprecipitation assays.

The present invention provides a sensitive and specific cell-based method for the detection of antibodies specifically produeed in AAG patients against subunits of the neuronal nicotinic acetylcholine receptors. The cells of the present invention are genetically engineered to express subunits of nAChRs, with or without the presence of other proteins (such as chaperons). Once the cells are in contact with sera from AAG patients, a binding is formed between the antibodies found in the sera and the transgenic nAChRs, which can be detected via a range of molecular and immunofluorescent techniques.

In a preferred embodiment of the present invention, the cells comprising the CBA of the present invention are HEK293 cells. However, other types of cells can be potentially used to carry out the present invention.

In yet another preferred embodiment of the present invention, the cells of the CBA express the α3 subunit of the nAChR. Without wishing to be bound by theory, the cells of the present invention may express other subunits of the nAChR, such as β₄, α₄, α₅, α₆, α₇ or β₂ subunits, individually or combined with other subunits, related to neurological autoimmune diseases.

In yet another preferred embodiment of the present invention, the cells of the CBA express in addition to subunits of nAChR, other proteins, such as chaperons. These chaperons may be, in a non-limiting way, chaperons associated with nicotinic acetylcholine receptors, such as NACHO or RIC-3.

In yet another preferred embodiment of the present invention, the cells may be cultured with substances or compounds which activate, trigger, inhibit or modify the activity or the expression levels of the nAChRs. Such materials might be for instance agonists or antagonists, such as nicotine, epibatidine, cytisine, varenicline, anatoxin-α, azetidine, altinicline, lobeline, epiboxidine, passetidine, alobelin, arecoline, metacholine, xanomeline, carbamylocholine, cevimeline, pilocarpine and more.

In yet another preferred embodiment of the present invention, the CBA is configured to detect relatively low titers of antibodies from AAG sera. It is well known in the art that some AAG patients manifest low anti-nAChR antibody titers, which makes it difficult to properly and accurately diagnose them (since those antibodies may appear in other neurological disorders). The CBA of the present invention is sufficiently sensitive to detect low titers, usually unnoticed or misinterpreted by other commonly used techniques, such as RIPA.

Detection of autoantibodies against nAChRs containing the α3-subunit (α3-nAChR/ganglionic nAChR) for the serological diagnosis of AAG has been usually performed up to now by a RIPA developed by Vernino & colleagues who have discovered the presence of α3-nAChR antibodies in AAG. It involves the use of detergent solubilized α3-AChR (from IMR32 cell extracts) preincubated with 125I-epibatidine which binds with high affinity to α3-nAChR. This assay is currently the method of choice for antibodies to ganglionic nAChR and, although it detects antibodies in only about 50% of the AAG patients, it has revolutionized AAG diagnosis, and therefore, the proper treatment of AAG. In addition, and in order to overcome the need for radioactivity, other researchers developed a luciferase immunoprecipitation system (LIPS) for the detection of antibodies that bind to individually expressed α3 or 4 AChR subunits (see Nakane et al., 2015).

While these assays have been invaluable for the correct diagnosis of AAG, they are not without caveats. Important limitations of their use in clinical practice include: (a) although the medium-high antibody titers are satisfactorily specific for AAG, about 50% of the low antibody titers seem to be non-specific, presenting in various mainly neurological diseases; (b) both assays, using detergent solubilized nAChR or its subunits, cannot discriminate between antibodies capable of binding to the cell-exposed intact nAChR (i.e. the potentially pathogenic) and antibodies to in vivo inaccessible nAChR sites: (c) RIPA can be performed only in a few laboratories eligible to use radioactivity, limiting thus the availability of the method: (d) LIPS uses individual nAChR subunits, which has the advantage of identifying the specific target-subunit (α3 or β4) but most probably they lack several epitopes, either due to non-native conformation of the individually expressed subunits or to the lack of the interphase between the subunits.

Over the last decade, it has been shown that CBAs for antibodies against cell surface antigens are superior methods for detecting autoantibodies of clinical significance. In CBAs, the detected antibodies bind to the extracellular epitopes of the antigens in native conformation, i.e. they are potentially pathogenic. Thus, CBAs are now the gold-standard assays for antibodies to several autoantigens involved in neuroimmune diseases especially for the detection of antibodies against cell-surface antigens.

However, until recently, the development of sensitive and reliable CBAs for antibodies against neuronal nAChRs was too difficult to be achieved because of low surface expression yield of these receptors. It has been shown that the chaperon RIC-3, and later the chaperon NACHO, increase the expression of some nAChR subtypes such as α7, α4β2, α384 and α3β2. In addition, the general nAChR agonist nicotine is also known to increase expression of some nAChR subtypes such as α7, α4β2, α334 and α3β2.

The major advantage of the novel CBA for α3-nAChR antibodies is its specificity for AAG. As described above, a disadvantage of the currently used assays is that they are not sufficiently specific for AAG, with about 50% of the patients with low (approximately between 0.05-0.2 nM) α3-nAChR antibody titers presenting with a variety of disorders other than AAG, including POTS, small and large-fiber neuropathy, LEMS, Hu-related paraneoplastic disorders and also non-neurological disorders, like Sjogren syndrome, systemic lupus erythematosus and autoimmune encephalopathy of unknown clinical significance. Therefore, the significance of a low RIPA-antibody titer is questionable.

### Example 1

To establish and evaluate the cell-based method of the present invention, the following materials were used and the following procedures and methods were condueted:

### Patients and Sera

Sera from two groups of patients were screened: (i) patients with neuroimmune diseases referred to Tzartos NeuroDiagnostics (Athens, Greece) between January 2017 and August 2020. Of them, 55 were referred for α3-nAChR antibody testing (by RIPA) and 2680 patients were referred for antibodies relevant to autoimmune encephalitis, paraneoplastic syndromes, neuromyelitis optica, autoimmune peripheral neuropathies and myasthenia gravis and Creutzfeldt Jakob disease; and (ii) 13 patients with autonomic failure who were positive for α3-nAChR antibodies following a radioimmunoprecipitation assay (RIPA-positive), identified in the Neurology Department, Carlo Besta Institute (Milan, Italy).

### Radioimmunoprecipitation Assay (RIPA)

The RIPA described in the present application was performed as previously described (see “Characterization of ganglionic acetylcholine receptor autoantibodies”, Vernino et al, 2008), with 125I-epibatidine labeled extracts of HEK293 cells transfected with α3β4 nAChR. Transfected HEK293 cells (see below) were solubilized with 0.5% Triton/PBS for 30 minutes at 4° C. Cell supernatant was incubated with ¹²⁵I-epibatidine for 1 hour at room temperature (RT). Indirectly 125I-labeled α3-nAChR (approximately 10,000 cpm/reaction) was incubated with patient's serum for 2 hours at RT, and subsequently overnight at 4° C. Then, goat anti-human IgG was added, incubated for 1.5 hours at 4° C., washed and radioactivity of the pellets was counted in a γ-counter. The cut-off for positivity was 0.05 nM (average of the values of 10 healthy controls+4SD). All sera tested by RIPA, were previously tested either in the diagnostic laboratory EuroDiagnostica, Sweden (the Athens sera, group (i)) or in Carlo Besta (the Milan sera, group (ii)) by the use of 125I-epibatidine labeled extracts of IMR32. All positive sera by the later system were also positive by the recombinant α384 nAChR, albeit with occasional differences in their titers.

### Cell Culture and Transfection

HEK293 cells were maintained in Dulbecco's modified Eagle's medium (DMEM) containing 10% fetal bovine serum (FBS) and 1% penicillin-streptomycin at 37° C. in 5% CO₂. Various parameters were tested in preliminary experiments to identify the optimal conditions. The final selected conditions involved the following: HEK293 cells were seeded on culture dishes and transiently transfected at 1:1:1:1 mixture of pCMV6-XL4-CHRNA3, pCMV6-XL5-CHRNB4 or pCMV6-XL5-CHRNB2, pcDNA-NACHO and pcDNA-RIC3 (Origene, Herford, Germany) or control vector using Jetprime kit transfection Reagent (Polyplus Jetprime, France). Cells were treated with ImM nicotine (N3876, Sigma), 24 hours before analysis. It is important to note that expression of most neuronal nAChRs via transfected cells is usually low, thus prohibiting the required high sensitivity of the corresponding CBAs. The chaperons RIC3 and NACHO, and the ligand nicotine, have been shown to increase expression of some nAChR subtypes (see: “NACHO Mediates Nicotinic Acetylcholine Receptor Function throughout the Brain”, Jose A. Matta et al, 2017).

### Development of the Novel Cell-Based Assay of the Present Invention

All sera were screened using the live-cell CBA with HEK293 cells expressing the α3β4 or α3β2 nAChR. 48 hours after the transfection, cells were washed with DMEM-0.46% w/v N-(2-hydroxyethyl) piperazine-N′-(2-ethanesulphonic acid) (HEPES) buffer (DMEM-HEPES buffer) in principle as previously described in the scientific literature (see “IgG1 antibodies to acetylcholine receptors in ‘seronegative’ myasthenia gravisy” Leite M I et al, 2008).

The CBA of the present invention involved an incubation of the tested sera at 1/10 dilution in 1% bovine serum albumin (BSA) in DMEM-HEPES buffer, with transfected cells. Following a 1-hour incubation at RT, cells were washed three times with DMEM-HEPES buffer and fixed immediately with 4% paraformaldehyde for 10 minutes at RT. Then, fixed cells were incubated with rabbit anti-human IgG (Invitrogen) at 1/750 dilution for 1 hour at RT, followed by an incubation with Alexa Fluor 568 goat anti-rabbit IgG (H+L) (Invitrogen), as third antibody, at 1/750 dilution for 1 hour at RT. As negative controls for all live-cell CBA-AQP4 transfected HEK293 cells were used. Positive sera were subsequently tested at serial dilutions to determine their titer of the sera expressed as the highest positive dilution. All positive sera were also tested with live CBA with HEK293 cells transfected with α4β2- and α7-nAChRs.

### Example 2

To identify sera which are positive for α3-nAChR antibodies using RIPA, two patient cohorts were tested for the presence of α3β4 nAChR antibodies in their sera: (i) 55 patients suspected for AAG referred from Greek Clinics to Tzartos NeuroDiagnostics; and (ii) a “confirmatory” group of 13 selected patients of Carlo Besta, Milan, already found positive in Milan by the RIPA with 125I-labeled IMR32 nAChR extracts and with verified final diagnosis.

In total, six out of the 55 patients of the Greek cohort referred for α3-nAChR antibodies, were found positive ( 6/55) by RIPA, 3 of whom presented high antibody titers (1.2-1.5 nM) and 3 presented low antibody titers (0.05-0.11 nM) (Table 1). In parallel, the following additional sera were also tested: 31 sera from patients positive for antibodies against voltage-gated Ca++-channels (VGCC) and 21 sera from patients positive for antibodies against the paraneoplastic antigen Hu; 4 and 2 sera respectively, were found to be RIPA-positive, all with low antibody titers, 0.06-0.11 nM (Table 1).

In addition, all 13 patients of the Milan cohort (group ii) were also found positive by the α3β4-nAChR RIPA; 11 with medium to high antibody titers (0.53-3.35 nM) and 2 with low antibody titers (0.05 and 0.13 nM) (Table 2).

TABLE 1
RIPA and CBA α3-nAChR antibody titers of all RIPA-positive patients
of the Greek cohort (group i) and summary of clinical characterization

| | | | Months | | | | | | RIPA titer |
|---|---|---|---|---|---|---|---|---|---|
| | | | with | | RIPA | CBA titer | CBA titer | | (nM) |
| Patient | | Age at | neurological | | titer | (α3β4 | (α3β2 | CBA ratio | needed for |
| no. | sex | onset | symptoms | Disease | (pmol/ml) | nAChR) | nAChR) | α3β4/α3β2 | α3β4 CBA+ |
| 1 | F | 70 | 24 | AAG | 1.40 | 1000 | 1000 | 1 | 0.014 |
| 2 | F | 67 | 4 | AAG | 1.17 | 300 | 300 | 1 | 0.039 |
| 3 | M | 54 | 2 | AAG | 0.83 | 1000 | 1000 | 1 | 0.008 |
| 4 | M | 35 | 48 | POTS | 0.11 | Neg | Neg | | |
| 5 | M | — | — | Inflammatory | 0.06 | Neg | Neg | | |
| | | | | myenteric | | | | | |
| | | | | ganglionopathy | | | | | |
| 6 | F | 47 | — | Autoimmune | 0.05 | Neg | Neg | | |
| | | | | sensory | | | | | |
| | | | | ganglionopathy | | | | | |
| | | | | (Sjögren) | | | | | |
| Average | | | | | | | | 1 | 0.020 |
| Patients with antibodies | | | | | | | | | |
| to Hu or VGCC | | | | Antigen | | | | | |
| 7 | — | — | — | Hu⁺ | 0.11 | Neg | Neg | | |
| 8 | — | — | — | VGCC⁺ | 0.10 | Neg | Neg | | |
| 9 | — | — | — | Hu⁺ | 0.08 | Neg | Neg | | |
| 10 | — | — | — | VGCC⁺ | 0.07 | Neg | NT | | |
| 11 | — | — | — | VGCC⁺ | 0.07 | Neg | Neg | | |
| 12 | — | — | — | VGCC⁺ | 0.06 | Neg | Neg | | |

TABLE 2
RIPA and CBA α3-nAChR antibody titers of all
RIPA-positive patients of the Italian cohort (group
ii) and summary of clinical characterization

| | | RIPA | | | CBA | RIPA titer |
|---|---|---|---|---|---|---|
| | | titer | CBA | CBA | ratio | (nM) |
| Patient | | (pmol/ | titer | titer | α3β4/ | needed for |
| no. | Disease | ml) | (α3β4) | (α3β2) | α3β2 | α3β4 CBA+ |
| 13 | AAG | 3.35 | 1000 | 250 | 4 | 0.034 |
| 14 | AAG | 3.22 | 1000 | 600 | 1.7 | 0.032 |
| 15 | AAG | 2.73 | 1000 | 250 | 4 | 0.027 |
| 16 | AAG | 2.20 | 1250 | 250 | 5 | 0.018 |
| 17 | AAG | 1.94 | 250 | 250 | 1 | 0.078 |
| 18 | AAG | 1.25 | 250 | 100 | 2.5 | 0.050 |
| 19 | AAG | 0.98 | 200 | 100 | 2 | 0.049 |
| 20 | AAG | 0.94 | 200 | 200 | 1 | 0.047 |
| 21 | AAG | 0.92 | 200 | 250 | 0.8 | 0.046 |
| 22 | AAG | 0.90 | 200 | 200 | 1 | 0.045 |
| 23 | AAG | 0.53 | 200 | 100 | 2 | 0.027 |
| 24 | POTS | 0.13 | Neg | Neg | | |
| 25 | AAG | 0.05 | 10 | Ambig- | >1 | 0.050 |
| | | | | uous | | |
| Average | | | | | 2.3 | 0.042 |

### Example 3

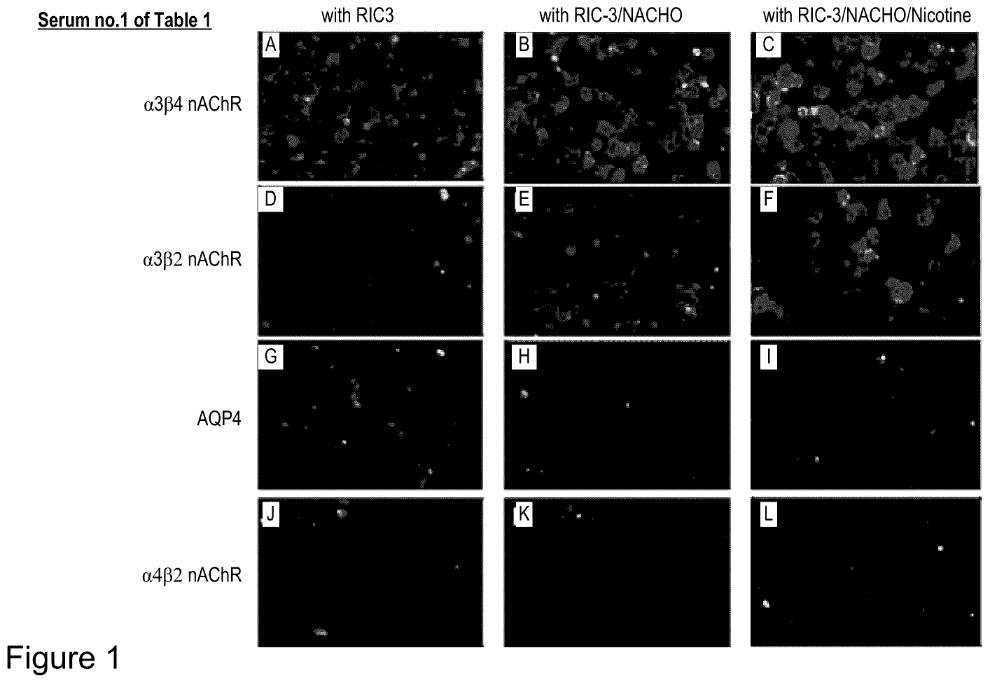

To further develop the sensitive CBA method of the present invention and optimize its conditions and parameters, two of the most strongly positive sera (later referred to as A and B) which were tested with RIPA for anti-α3 antibodies and an anti-β2 monoclonal antibody were used to determine the optimal conditions for the development of a highly sensitive and accurate CBA. The expression of α384 and α32 nAChRs was compared under 4 different conditions, all of which in the presence of the RIC3 chaperon: (a) co-expression with the NACHO chaperon and culture with 1 mM nicotine 24 hours after transfection; (b) only co-expression with NACHO; (c) only culture with 1 mM nicotine; and (d) neither. (results are also depicted in Table 3 and FIG. 1).

Concluding the above-described assay, the combined use of both chaperons (RIC-3 and NACHO) and 1 mM nicotine results in the strongest staining/signal for both α384 and α3β2 nAChRs. Therefore, the inventors adopt the use of all 3 factors in all subsequent experiments disclosed in the present application. Similarly, the use of these factors (at different concentrations) was also found beneficial for the expression of the α432 subunit. None of the Greek or Italian cohorts of patients that were suspected for autonomic failure had antibodies for α4β2. However two patients with autoimmune encephalopathy were positive for a4b2 antibodies.

TABLE 3
Calibration and optimization of conditions for the
α3β4-nAChR CBA of the present invention

| | CBA with HEK293 cells transfected with | | | |
|---|---|---|---|---|
| | α3β4 nAChR + RIC-3 chaperon and: | | | |
| Sera | — | NACHO | Nicotine in | NACHO + Nicotine |
| | | | the culture | in the culture |
| NHS | Negative | Negative | Negative | Negative |
| (Normal | | | | |
| human | | | | |
| serum) | | | | |
| A | 1+ | 3+ | 3+ | 3+ |
| B | Negative | 1+ | 1+ | 2+ |

Legend:
(1+) - weakly positive,
(3+) - strongly positive

Reference is now made to FIG. 1, depicting immunofluorescence images of the CBA of the present invention under different conditions. HEK293 cells were transfected with α3β4 and α3β2 nAChRs (or α4β2 and AQP4 as controls), with the chaperon RIC-3, or RIC-3 and NACHO, or also treated with nicotine. Then, all transfected cells were incubated with a serum from an AAG patient with high α3 titer, and stained with anti-human IgG (red).

Image 1A depicts the immunofluorescence emitted from cells transfected with RIC-3 and α3β4 nAChR.

Image 1B depicts the immunofluorescence emitted from cells transfected with RIC-3, NACHO and α3β4 nAChR.

Image 1C depicts the immunofluorescence emitted from cells transfected with RIC-3, NACHO and α384 nAChR, also cultured with nicotine.

Image 1D depicts the immunofluorescence emitted from cells transfected with RIC-3 and α3β2 nAChR.

Image 1E depicts the immunofluorescence emitted from cells transfected with RIC-3, NACHO and α3β2 nAChR.

Image 1F depicts the immunofluorescence emitted from cells transfected with RIC-3, NACHO and α3β2 nAChR, also cultured with nicotine.

Image 1G depicts the immunofluorescence emitted from cells transfected with RIC-3 and negative control AQP4.

Image 1H depicts the immunofluorescence emitted from cells transfected with RIC-3, NACHO and negative control AQP4.

Image 1I depicts the immunofluorescence emitted from cells transfected with RIC-3, NACHO and negative control AQP4, also cultured with nicotine.

Image 1J depicts the immunofluorescence emitted from cells transfected with RIC-3 and α4β2 nAChR.

Image 1K depicts the immunofluorescence emitted from cells transfected with RIC-3, NACHO and α4β2 nAChR.

Image 1L depicts the immunofluorescence emitted from cells transfected with RIC-3, NACHO and α4β2 nAChR, also cultured with nicotine.

As can be clearly seen from the images depicted in FIG. 1, the combination of co-expression of the RIC-3 and NACHO chaperons with the addition of nicotine to the cell culture 24 hours after transfection, the expression of the autonomic ganglionic nAChR subunits: α3β4 and α3β2 is significantly increased (FIG. 1C and FIG. 1F), allowing sufficient antibody binding, thus generating a sensitive CBA specifically for AAG patients.

### Example 4

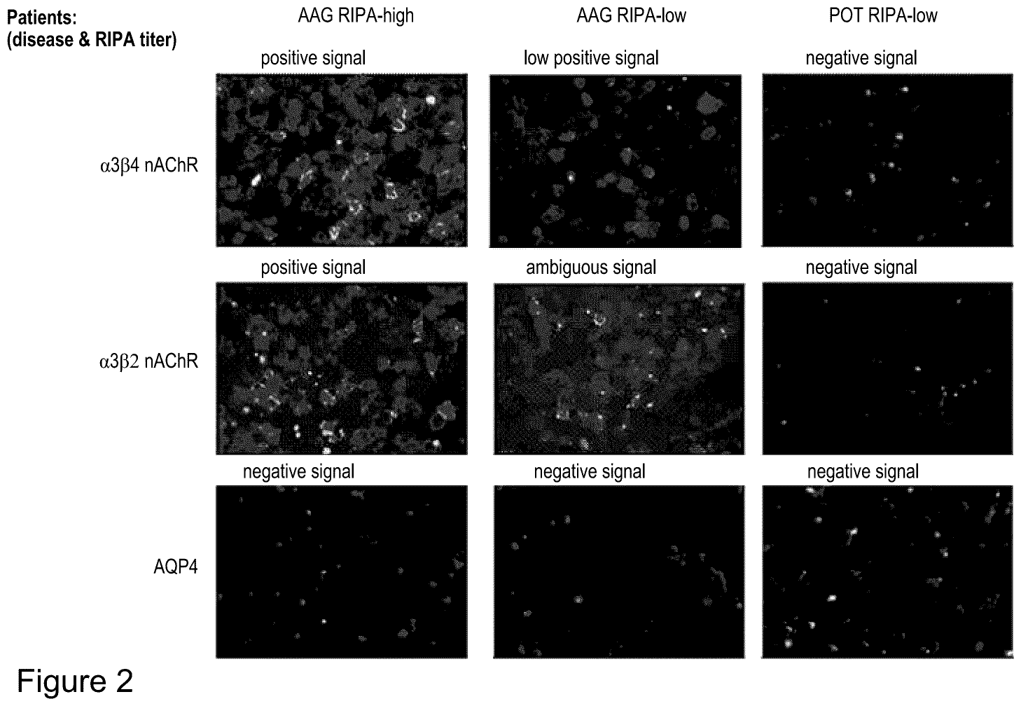

Reference is now made to FIG. 2 showing immunofluorescence images of the binding of selected sera to α3β4 and α3β2 nAChRs but not to the control (APQ4) transfected HEK293 cells.

The novel CBA of the present invention is based on α384 and α3β2 nAChR transfected HEK293 cells, which detect antibodies in sera with high or low RIPA-titers, but only and specifically in AAG patients.

HEK293 cells expressing α334, α3β2 or AQP4 were incubated with sera from AAG patients (of high or low RIPA titer) or from a POTS (postural orthostatic tachycardia syndrome) patient, and stained with anti-human IgG (red). It is shown that both AAG patients gave positive staining, independently of the RIPA titer, whereas the POTS patient gave no staining.

### Example 5

To properly compare the commonly used technique RIPA to the novel CBA of the present invention for the purpose of detecting and diagnosing AAG patients based on the serum antibodies, the following assay was condueted:

Using the live CBAs with α384 and α3β2 nAChRs, with the best above identified conditions, all sera of the Greek cohort were screened, including the 12 RIPA-positive sera from the Greek cohort. As controls, sera from 2680 patients suspected for other neuroimmune diseases (referred for the detection of antibodies to other neurological antigens) were screened, including 100 sera from healthy individuals or patients with neurodegenerative diseases, Alzheimer and CJD and the 46 RIPA-negative sera from patients with VGCC or Hu antibodies. As shown in Table 1 (column: CBA titer). 3/12 RIPA-positive patients were found α3-nAChR CBA-positive. The 3 CBA-positive patients suffered from AAG, whereas none of the RIPA-positive/CBA-negative patients suffered from AAG.

Subsequently, the sera of the “confirmatory” Italian cohort of 13 RIPA-positive patients were tested. 12/13 patients were found CBA-positive. All 12 patients suffered from AAG, whereas the CBA-negative patient had POTS (Table 2).

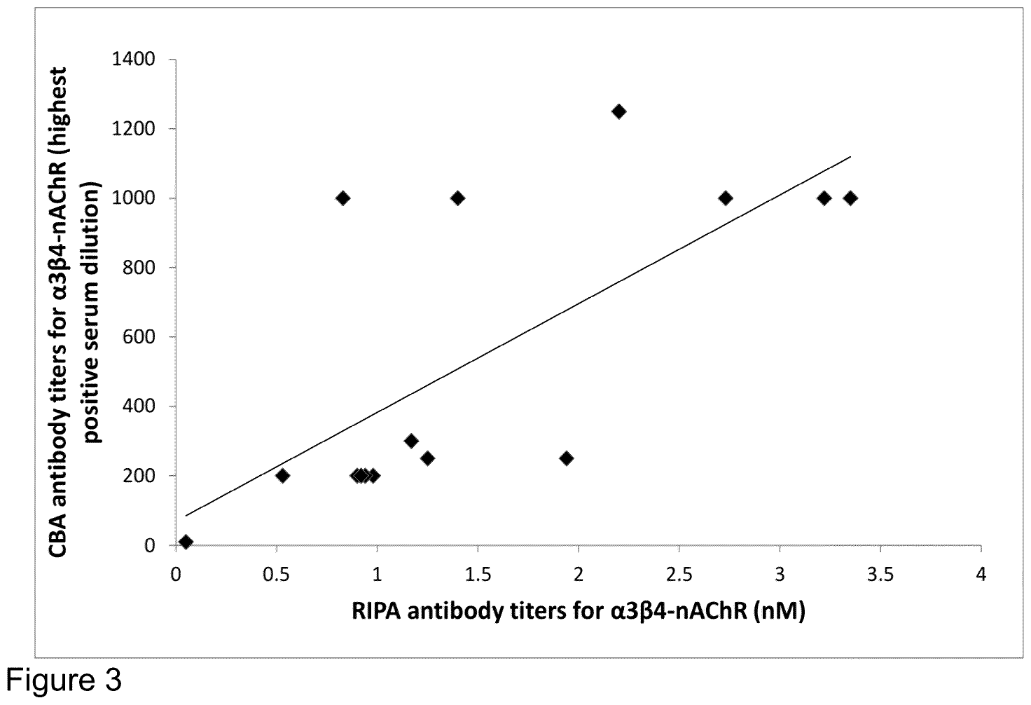

Reference is now made to FIG. 3 graphically showing the correlation between RIPA titer and CBA titer (maximum serum dilution for positive staining). Said correlation exists, but it is rather weak.

Table 1 and Table 2 (CBA columns) show that while half of the CBA-positive sera had similar CBA titer for both α3β4 and α3β2 nAChRs, the other half had higher titers with the α3β4 nAChR. On average, CBA with α384 nAChR gave nearly 2 times higher titers than CBA with α3β2 nAChR but with significant variations between the sera: from 0.8-5.0 times higher. Yet, almost all sera positive for α384 nAChR were also positive for α3β2 nAChR, which suggests that all sera contain anti-α3 antibodies, with or without anti-β2 or -β4 antibodies. The 14 of these patients had moderate to high RIPA titers (0.5-3.2 nM), whereas one patient had borderline RIPA titer (0.05 nM).

Although most CBA-positive sera had high RIPA titers, and all CBA-negative sera had low RIPA titers, apparently the antibody concentration was not the limiting factor for CBA-positivity, because CBA could efficiently detect low concentrations of antibodies in the sera of AAG patients. The right column of Table 1 and Table 2 (“RIPA titer (nM) needed for α3β4 CBA+”) present the calculated minimum RIPA titer for each test serum which would be needed for positive CBA (at the adopted 1/10 dilution). These minimum RIPA titers required for CBA positivity, are derived from the following ratio: (RIPA titer)/(CBA titer) multiplied by 10. It is shown that average RIPA titers 0.020 and 0.042 nM for the Greek and Italian cohorts (total: 0.038+0.018 nM) would be sufficient for CBA positivity. Taking into account that the cut-off for positivity of the α3-nAChR RIPA is 0.05 nM, the inventors thus conclude that CBA is marginally more sensitive than RIPA (the commonly used technique for detecting antibodies in AAG patients).

### Example 6

Clinical Characterization of Patients with α3-nAChR Antibodies (with RIPA and/or CBA) Shows Superiority of the CBA, with High Specificity to AAG

Clinical characterization of the 19/25 RIPA-positive patients showed that 15 of them had AAG, whereas the remaining 4 patients had other neurological diseases (see Table 1 and Table 2). However, in terms of RIPA titer, all 14 patients with medium-high α3-nAChR antibody titer had AAG, in comparison to only one of the 5 low titer patients who had AAG. Among the remaining 4 low RIPA titer patients: 2 had postural orthostatic tachycardia syndrome, one had autoimmune sensory neuropathy with Sjögren disease and one had inflammatory myenteric ganglionopathy.

Interestingly, the novel CBA of the present invention, opposed to RIPA, detected α3-AChR antibodies selectively in all 15 AAG patients, independently of high or low RIPA titer, but in none of the 4 non-AAG patients, despite the fact that they were detected as positive by RIPA. In addition, as shown above, none of the 6 RIPA-positive patients identified from the groups of patients with VGCC or Hu antibodies was CBA-positive.

Finally, the inventors used the α3-nAChR CBA of the present invention (with α384 and α3β2 nAChRs) to test sera from 2644 patients with other verified or suspected neurological diseases (including autoimmune encephalitis, paraneoplastic syndromes, NMO, myasthenia gravis, thymoma, Creutzfeldt Jakob etc.), all of which were found CBA-negative.

### Example 7

Ig Class and Subclass of the α3-nAChR Antibodies Determined by the Novel CBA of the Present Invention

The Ig class and subclass of the α3-nAChR antibodies were determined, since this characteristic is likely to be related to their pathogenicity. The Ig class/subclass of the AAG-relevant antibodies was specifically determined, i.e. by the use of the CBAs for α3β4 nAChR. Table 4 shows that all tested CBA-positive sera contain α3-nAChR antibodies of the complement-binding IgG1 subtype. In addition, one serum also contained IgG2 and IgG3 antibodies, whereas another two contained either IgG2 or IgG3 antibodies. Finally, one additional serum contained IgM antibodies (in addition to IgG1).

TABLE 4
Immunoglobulin class and subclass of the CBA-positive sera

| | Ig class, subclass* | | | | |
|---|---|---|---|---|---|
| | IgG1 | IgG2 | IgG3 | IgG4 | IgM |
| Patient | CBA for α3β4 | | | | |
| 1 | + | − | − | − | − |
| 2 | + | − | + | NT | − |
| 3 | + | − | − | − | − |
| 13 | + | − | − | − | − |
| 14 | + | − | − | − | − |
| 15 | + | + | − | − | − |
| 16 | + | − | − | − | − |
| 17 | + | − | − | − | − |
| 18 | + | − | − | − | − |
| 19 | + | − | − | − | − |
| 20 | + | − | − | − | − |
| 21 | + | − | − | − | + |
| 22 | + | + | + | − | − |
| 23 | + | − | − | − | − |

*+: positive,
−: negative,
?: ambiguous,
NT: not tested

In conclusion, it was observed that the CBA of the present invention is of higher sensitivity than the established RIPA, as it is AAG-specific (100% in the results disclosed herein). Specifically, all AAG patients were both RIPA and CBA-positive. Although the majority of these patients had medium to high RIPA titers (which are considered AAG-specific), one patient had a borderline RIPA titer (0.05 nM), and yet was also CBA-positive. In contrast, all 4 non-AAG patients with low RIPA titers (0.05-0.13 nM) were CBA-negative.

### Example 8

Detection of Antibodies to α482 nAChR in Autoimmune Encephalitis Patients with α4β2 nAChR CBA.

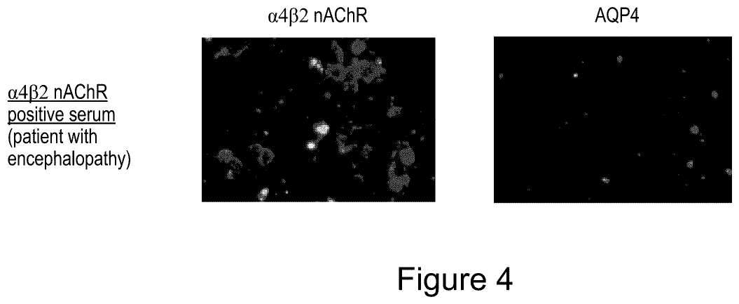

Applying the same conditions as for α3β4 nAChR expression (including the two chaperons and nicotine), HEK293 cells were also transfected and hyper-expressed α4β2 nAChR. CBA with these cells, although it was found negative for the AAG sera (as described in example 3), was found positive for sera from two patients of the spectrum autoimmune encephalitis disorders, which gave negative CBA with the other AChRs (α3β4, α3, β2, α7), concluding that the antibodies bind to the α4 subunit. Specifically, it concerns the patient who had Rasmussen autoimmune encephalitis and the patient who had autoimmune miningoenchephalomyelitis. The results are also depicted in FIG. 4.

Based on the reported presence of low α3-nAChR antibody titers in patients with LEMS and antibodies to VGCC and in patients with the paraneoplastic anti-Hu antibodies, the inventors searched for α3-nAChR antibodies, using RIPA and CBA, in 52 patients positive for antibodies to VGCC or Hu. Six patients were found RIPA-positive (0.06-0.11 nM) but none was CBA-positive. Apparently, the low antibody concentration of the 10 RIPA-positive patients was not the reason for CBA-negative result, because (a) the AAG patient with the even lower, borderline, RIPA titer (0.05 nM) was CBA-positive; and (b) based on the maximum CBA-positive dilutions of the 15 AAG patients the inventors calculated that if these patients had as low as 0.038 nM RIPA titers (i.e. below the RIPA cutoff) they would still be CBA-positive. Since the generally used cut-off RIPA titer is 0.05 nM, it is concluded that the CBA is at least as sensitive (if not more sensitive) as the RIPA: however, it is positive only for antibodies specific to AAG patients.

All sera of Table 1 and Table 2 were tested with CBA with both ganglionic nAChR subtypes, α3β4 and α3β2. All sera bound to both nAChRs, strongly suggesting that all had antibodies to the α3 subunit. This is further supported by the fact that none of these sera bound to the α4β2 nAChR, therefore, any binding to α32 nAChR should be due to binding to the α3-subunit. Nonetheless, there was very considerable variation in the titer ratio between α3β4 and α3β2 nAChRs (serum dilutions for positive CBA with α3β4 versus α3β2 nAChR) among the different sera, from 0.8 to 5 (average 1.9). The high α3β4/α3β2 ratios ( 6/14 sera with ratio≥2:3 sera with ratio 4-5) could be attributed to several possible reasons, including: (a) that some sera may also contain anti-β antibodies, especially anti-β4: (b) several antibodies may bind on the interphase between the α3 and β2 or β4 subunits while β2 and β4 should probably form different epitopes in their interphases with the @3-subunit; or (c) the two different β-subunits may impose different conformational changes on the α3 epitopes. Although the presence of anti-β4 antibodies in some sera is likely, as it has been shown by Nakane et al. (2015, 2018), in order to justify the much higher titers for α3β4 nAChR than for α3β2 nAChR in several sera, the anti-β4 antibodies should be of much higher concentrations than the anti-α3 antibodies. However. Nakane et al. (2015) showed that anti-β4 antibodies are generally much fewer than the anti-α3 antibodies, which does not support the possibility of predominance of anti-β4 over anti-α3 antibodies. Any possible difference in expression efficiency between the two nAChRs is unlikely to be the reason for the different CBA titers because such a difference would be expected to have similar effect on all sera. The inventors suggest that differences in subunit interphases and conformational differences on the α3 subunits between the two nAChRs are the reasons for the different CBA titers between the two nAChRs.

In summary, the present examples disclosed in this application suggest that the novel CBA for the detection of α3-nAChR antibodies, is marginally more sensitive than the currently established RIPA, but, more importantly, it is highly AAG-specific contrary to the RIPA which is not sufficiently AAG-specific in the low antibody titers, even at the lowest titers. Furthermore, in comparison to RIPA which can be performed only in the few diagnostic laboratories eligible to use radioactivity, this CBA, following the above-mentioned conditions for high α3-nAChR expression, can be easily performed by many laboratories worldwide.

1.-38. (canceled)

39. A cell-based method for detecting antibodies against α3 subunits of the neuronal nicotinic acetylcholine receptors in a subject, comprising steps of:
a. engineering cells to express
(i) α3 subunit of the neuronal nicotinic acetylcholine receptors (nAChR), together with a β subunit (β2 or β4) necessary to form the intact α3-containing nAChR and
(ii) at least one chaperon;
b. culturing said cells with at least one substance;
c. exposing said cells to body fluid or tissue from said subject; and
d. detecting a reaction between said at least one protein and said antibodies found in said body fluid or tissue,
thereby, obtaining a diagnosis of autoimmune autonomic ganglionopathy (AAG) for said patient.

40. The method of claim 39, wherein said antibodies against α3 subunits of neuronal-type nicotinic acetylcholine receptors are selected from a group consisting of IgG, IgA, IgM and any combination thereof.

41. The method of claim 39, wherein said subject suffers a neurological disorder.

42. The method according to claim 39, wherein said at least one chaperon is RIC-3, NACHO, or both.

43. The method of claim 39, wherein said engineering is conducted by means of molecular biology or genetic engineering.

44. The method of claim 39, wherein said cells are selected from a group consisting of rodent cells, simian cells, human cells, diseased cells, transgenic cells and any combination thereof.

45. The method of claim 39, wherein said at least one substance is a substance configured to activate, trigger, inhibit or modify the activity or the expression levels of said α-3 subunit.

46. The method of claim 45, wherein said at least one substance is selected from a group consisting of nicotine, acetylcholine, epibatidine, cytisine, varenicline, anatoxin-α, azetidine, altinicline, lobeline, epiboxidine, passetidine, alobelin, arecoline, metacholine, xanomeline, carbamylocholine, cevimeline, pilocarpine and any combination thereof.

47. The method of claim 39, wherein said body fluid or tissue selected from a group consisting of blood, serum, cerebrospinal fluid, lymph, urine, sweat, saliva and any combination thereof.

48. The method of claim 39, wherein said detecting a reaction is conducted by means of molecular biology, immunofluorescence, radioactivity, luminescence and any combination thereof.

49. A kit for detecting antibodies against α-3 subunits of the nicotinic acetylcholine receptors in a subject, comprising:
a. cells expressing
(i) α3 subunits of the nicotinic acetylcholine receptors, together with a β subunit (β2 or β4) necessary to form the intact α3-containing nAChR.
(ii) at least one chaperone
b. at least one medium for cell culture,
c. at least one buffer for cell culture; and
d. at least one substance

50. The kit of claim 49, wherein said cells are selected from a group consisting of rodent cells, simian cells, human cells, diseased cells, transgenic cells and any combination thereof.

51. The kit of claim 49, wherein said at least one substance is a substance configured to activate, trigger, inhibit or modify the activity or the expression levels of said α-3 subunit.

52. The kit according to claim 51, wherein said at least one substance is selected from a group consisting of nicotine, acetylcholine, epibatidine, cytisine, varenicline, anatoxin-α, azetidine, altinicline, lobeline, epiboxidine, passetidine, alobelin, arecoline, metacholine, xanomeline, carbamylocholine, cevimeline, pilocarpine and any combination thereof.

53. The kit of claim 49, wherein said cells are fixed with methanol, paraformaldehyde or other fixatives.

54. A cell carrying at least one expression vector for use in detecting of antibodies against α-3 subunit of the nicotinic acetylcholine receptors in a subject.

55. The cell according to claim 54, wherein said at least one expression vector encodes for the expression of
(i) α-3 subunits of the nicotinic acetylcholine receptors
(ii) at least one chaperon

56. The cell according to claim 54, wherein said chaperon is RIC-3, NACHO, or both.
